(19) United States
(12) Patent Application Publication
(10) Pub. No.: US 2020/0046276 A1
(43) Pub. Date: Feb. 13, 2020

(54) ARTERIAL SYRINGE ASSEMBLY

(71) Applicant: Vyaire Medical Consumables LLC, Yorba Linda, CA (US)

(72) Inventor: Anthony VALENTINO, Corona, CA (US)

(21) Appl. No.: 16/485,525

(22) PCT Filed: Feb. 16, 2018
(86) PCT No.: PCT/US2018/000045

§ 371 Date: Aug. 13, 2019

Related U.S. Application Data
(60) Provisional application No. 62/460,664, filed on Feb. 17, 2017.

(51) Int. Cl. A61B 5/15; A61B 5/153

(52) U.S. Cl. CPC A61B 5/150664; A61B 5/150236; A61B 5/150213; A61B 5/153

(57) ABSTRACT

An arterial syringe assembly is disclosed. The assembly includes a housing with a proximal end that may couple with a syringe barrel and a distal end that may couple with a needle. A needle cover is coupled with the housing at a hinge that may rotate to position the needle cover relative to the needle and may rotate to an orientation aligned with the needle such that the needle is within the needle cover. A needle plug is coupled to the needle cover at a distal end of the needle cover and may include plug material. The needle plug may slide axially relative to the needle to advance the needle plug at a distal end of the needle. A hydrophobic filter is positioned within the syringe barrel to evacuate air through a proximal end of the syringe barrel after the needle tip extends within the plug material.

### CROSS-REFERENCE TO RELATED APPLICATIONS

This application is the national stage of International Patent Application No. PCT/US2018/000045, filed Feb. 16, 2018, which claims the benefit of priority under 35 U.S.C. § 119 from U.S. Provisional Patent Application 62/460,664 filed Feb. 17, 2017, which is incorporated herein by reference in its entirety.

### FIELD OF THE INVENTION

The present invention generally relates to syringe assemblies and more particularly to syringe assemblies for drawing arterial blood.

### BACKGROUND

Arterial blood samples are collected from an artery to determine, for example, arterial blood gas levels. A caregiver obtains a sample using a needle and syringe or through a catheter. Inaccurate samplings may result from a number of factors, including the presence of air in the sample. Accordingly, some arterial syringes are prepared to reduce the amount of air the blood sample may be exposed to when the blood is drawn.

### BRIEF DESCRIPTION OF THE DRAWINGS

For a more complete understanding of the present disclosure, and the advantages thereof, reference is now made to the following descriptions to be taken in conjunction with the accompanying drawings describing specific aspects of the disclosure, wherein:

FIG. 1 illustrates an example arterial syringe assembly, according to some aspects of the disclosure.

FIG. 2A illustrates a side view of an example arterial syringe assembly, according to some aspects of the disclosure.

FIG. 2B illustrates a bottom view of an example arterial syringe assembly, according to some aspects of the disclosure.

FIG. 3A illustrates an example arterial syringe assembly with a needle locked and ready for shut off, according to some aspects of the disclosure.

FIG. 3B illustrates the fully locked and shut off configuration of an example arterial syringe assembly, according to some aspects of the disclosure.

FIG. 4A illustrates an interior view of an example arterial syringe assembly, according to some aspects of the disclosure.

FIG. 4B illustrates a top view of an example arterial syringe assembly, according to some aspects of the disclosure.

FIG. 4C illustrates a detailed view of locking tabs of the example arterial syringe assembly of FIG. 4A, according to some aspects of the disclosure.

FIG. 4D illustrates a side view of an example arterial syringe assembly having an inner sleeve and an outer sleeve, according to some aspects of the disclosure.

FIG. 4E illustrates a cross sectional view of a distal end of an example arterial syringe assembly, according to some aspects of the disclosure.

FIG. 5A illustrates a top view of components of an example arterial syringe assembly, according to some aspects of the disclosure.

FIG. 5B illustrates a side view of components of an example arterial syringe assembly, according to some aspects of the disclosure.

FIGS. 6A-6D illustrate components of a hinge of an example arterial syringe assembly, according to some aspects of the disclosure.

FIGS. 7A-7D illustrate components of an example arterial syringe assembly, according to some aspects of the disclosure.

FIG. 8A-8D illustrate components of an example arterial syringe assembly, according to some aspects of the disclosure.

FIG. 9 illustrates a flow diagram of an example process of operating an arterial syringe assembly, according to some aspects of the disclosure.

FIG. 10A illustrates a side view of an example arterial syringe assembly, according to some aspects of the disclosure.

FIG. 10B illustrates a side view of an example arterial syringe assembly, according to some aspects of the disclosure.

FIGS. 11A-11B illustrate side views of arterial syringe assembly of FIG. 10A with the needle cover in a fully open position, according to some aspects of the disclosure.

FIG. 11C illustrates a side cutaway view of the arterial syringe assembly of FIG. 10A, according to some aspects of the disclosure.

FIG. 11D-11E illustrate side views of arterial syringe assembly of FIG. 10A, according to some aspects of the disclosure.

FIG. 11F illustrates arterial syringe assembly of FIG. 10A in the locked position, according to some aspects of the disclosure.

FIG. 12A-12B illustrate side views of arterial syringe assembly of FIG. 10A having the needle cover in a locked position, according to some aspects of the disclosure.

FIG. 12C illustrates a side view of arterial syringe assembly of FIG. 10A in the locked position, according to some aspects of the disclosure.

FIGS. 13A-13B illustrate side views of an arterial syringe assembly, according to some aspects of the disclosure.

FIGS. 14A-16B illustrate side views of an arterial syringe assembly, according to some aspects of the disclosure.

### SUMMARY

In many instances of arterial blood sampling, it is desirable not to add or permit air to remain in the blood sample after the blood sample is drawn from the patient. Air in the blood sample, for example, may hinder the ability to accurately measure the oxygen content of the patient's blood sample. However, air bubbles may be commingled with the blood sample during or after the time when the blood sample is collected from the artery. Approaches for minimizing contact of air with the blood sample are desirable.

According to various aspects of the subject technology, an arterial syringe assembly is described. The arterial syringe assembly includes a housing including (i) a proximal end, (ii) a distal end, and (iii) a hinge. The proximal end may couple with a distal end of a syringe barrel. The distal end of the housing may couple with a needle. The arterial syringe assembly includes a needle cover coupled with the housing at the hinge that may rotate about the hinge to position the needle cover relative to the needle and may rotate to an orientation that is axially aligned with the needle such that the needle is received within the needle cover. The arterial syringe assembly includes a needle plug coupled to the needle cover at a distal end of the needle cover that includes a plug material. The needle plug may slide axially relative to the needle cover and the needle to advance proximally within the needle cover when the needle cover is axially aligned with the needle such that a tip of the needle extends within the plug material. The arterial syringe assembly includes a hydrophobic filter positioned within the syringe barrel to evacuate air through a proximal end of the syringe barrel after the needle tip extends within the plug material.

According to various aspects of the subject technology, an arterial syringe assembly is described. The arterial syringe assembly includes a housing including (i) a proximal end, (ii) a distal end, and (iii) a hinge. The proximal end may couple with a distal end of a syringe barrel. The distal end of the housing may couple with a needle. The arterial syringe assembly includes a needle cover coupled with the housing at the hinge and that may rotate about the hinge to position the needle cover relative to the needle and to rotate to an orientation that is axially aligned with the needle such that the needle is received within the needle cover. The arterial syringe assembly includes a needle plug coupled to the needle cover at a distal end of the needle cover and on an outer surface of the needle cover. The needle plug includes a plug material. The needle plug may slide along the outer surface of the needle cover to advance proximally within the needle cover when the needle cover is axially aligned with the needle such that a tip of the needle extends within the plug material. The arterial syringe assembly includes a hydrophobic filter positioned within the syringe barrel to evacuate air through a proximal end of the syringe barrel after the needle tip extends within the plug material.

According to various aspects of the subject technology, a method of operating an arterial syringe assembly is described. The method includes providing an arterial syringe assembly including providing an arterial syringe assembly comprising (i) a proximal end may couple with a distal end of a syringe barrel, (ii) a distal end coupled to a needle, (iii) a hinge coupled with a needle cover, and (iv) a needle plug, which holds a plug material, coupled with the needle cover at a distal end of the needle cover. The method includes rotating the needle cover about the hinge to position the needle cover relative to the needle and to rotate to an orientation that is axially aligned with the needle such that the needle is received within the needle cover. The method includes sliding the needle plug axially relative to the needle cover. When the needle cover is axially aligned with the needle, to advance the needle plug proximally relative to the needle, such that a distal end of the needle extends within and is plugged by the plug material. The method includes evacuating, via a hydrophobic filter positioned within the syringe barrel, air through a proximal end of the syringe barrel after the needle tip extends within the plug material.

The foregoing has outlined rather broadly the features of the present disclosure in order that the detailed description that follows can be better understood. Additional features and advantages of the disclosure will be described hereinafter, which form the subject of the claims.

### DETAILED DESCRIPTION OF THE PREFERRED EMBODIMENTS

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, it will be clear and apparent to those skilled in the art that the subject technology is not limited to the specific details set forth herein and can be practiced using one or more implementations. In one or more instances, well-known structures and components are shown in block diagram form in order to avoid obscuring the concepts of the subject technology.

According to some implementations, the subject technology provides an arterial syringe assembly that evacuates air from the syringe barrel after the blood or fluid sample is collected in the needle. In brief, the subject technology relates to an arterial syringe assembly that includes a housing that has a proximal end, a distal end, and a hinge. The proximal end accepts a syringe plunger or a syringe barrel, and the distal end accepts a needle. A needle cover is coupled with the housing at the hinge. The needle cover may rotate about the hinge to position the needle cover relative to the needle. The needle cover may rotate to an orientation that is axially aligned with the needle such that the needle is received within the needle cover. A needle plug, which includes a plug material, is coupled to the needle cover at a distal end of the needle cover. The needle plug is configure to slide axially relative to the needle cover and the needle to advance proximally with the needle cover when the needle cover is axially aligned with the needle such that a tip of the needle extends within the plug material. A hydrophobic filter is positioned within the syringe barrel to evacuate air through a proximal end of the syringe barrel after the needle tip extends within the plug material. In some aspects, because of the operation of the hydrophobic filter, air leaves the syringe barrel, while blood or fluid remains within the syringe barrel.

The arterial syringe assembly may be operated single-handedly. A caregiver using the arterial syringe assembly may operate the arterial syringe assembly, for example to draw and store blood or fluid, in one hand while having his/her other hand free, for example, to hold a bandage and to apply the bandage to the patient.

In some implementations, the needle plug, which includes the plug material, is coupled to the needle cover at a distal end of the needle cover and on an outer surface of the needle cover. The needle plug slides along the outer surface of the needle cover to advance proximally within the needle cover when the needle cover is axially aligned with the needle such that a tip of the needle extends within the plug material.

According to some examples, a locking tab is provided on the needle cover. The locking tab engages the needle plug. With the application (e.g., by a caregiver) of sufficient force, the tab is lifted out of the detent of the needle plug. The tab then rides along the channel between two detents of the needle plug, until the tab extends within the second detent. At this position, the needle has advanced into the plug material that occludes the distal tip of the needle. With the needle tip occluded, a force can be applied against the syringe plunger that will cause the air within the syringe barrel to pass through the hydrophobic filter and be released through a proximal end of the syringe barrel. In these circumstances, air will not escape through the needle tip.

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

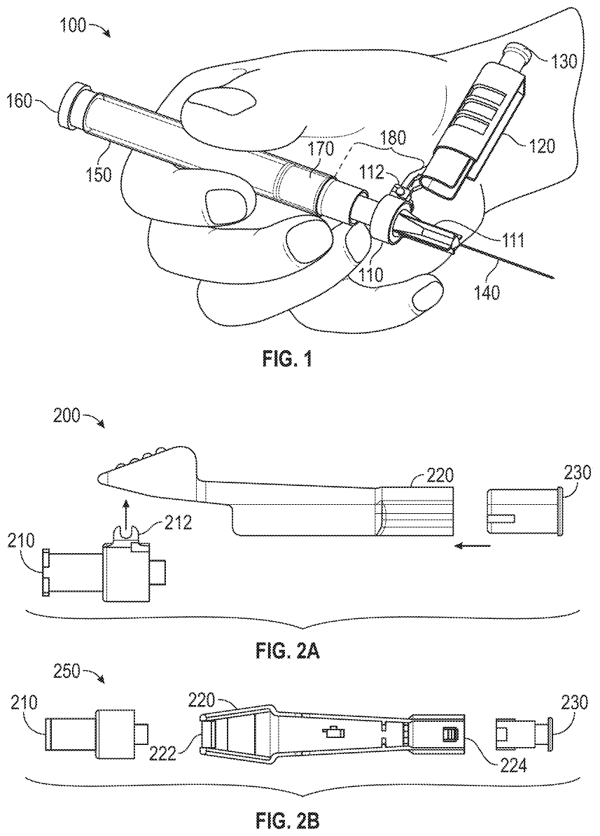

FIG. 1 illustrates an example arterial syringe assembly according to some aspects of the disclosure. As shown, arterial syringe assembly 100 includes luer adapter 110, needle cover 120, needle plug 130, needle 140, syringe barrel 150, plunger 160, and hydrophobic filter 170. Arterial syringe assembly 100 may be operated single handedly, with a caregiver holding and operating the device in a single hand and having the second hand free for other activities, such as applying a bandage to a patient or for pressing on the artery.

In some embodiments, the caregiver inserts needle 140 through the patient's skin into the patient's artery to draw blood or fluid from the artery. The blood or fluid is drawn, or permitted to flow, from the artery into syringe barrel 150. In some examples, plunger 160 is withdrawn by the caregiver while drawing blood or fluid. In some examples, plunger 160 is not withdrawn by the caregiver while drawing blood or fluid. The arterial blood pressure may be sufficient to cause the syringe barrel to be filled with blood. After the blood or fluid has filled syringe barrel 150 to a predetermined level, the caregiver may seal or occlude the distal end of needle 140, as described herein, such that blood or air does not flow out of the needle. The caregiver may push plunger 160 in distal direction to expel air from syringe barrel 150. In some embodiments, hydrophobic filter 170 is positioned within syringe barrel 150 at the distal end of plunger 160 and may be coupled with the distal end of the plunger. Hydrophobic filter 170 is used to evacuate air through a proximal end of syringe barrel 150 when the caregiver pushes the plunger in the distal direction. In some embodiments, by using hydrophobic filter 170, air may be evacuated from the interior of syringe barrel 150 to the atmosphere or ambient environment outside of arterial syringe assembly 100. In some examples, hydrophobic filter 170 constrains the blood or fluid within the interior of syringe barrel 150 to prevent the blood or fluid from evacuating the interior of syringe barrel 150 together with the air.

In some embodiments as shown in FIG. 1, luer adapter 110 connects needle 140, the syringe barrel 150, and needle cover 120. Hinge portion 112 adjoins luer adapter 110 and needle cover 120. After the caregiver finishes drawing blood or fluid from the patient's artery into syringe barrel 150, the caregiver may rotate needle cover 120 relative to luer adapter 110 about hinge portion 112. The rotation may make the needle cover 120 approximately parallel with needle 140 and the central axis of syringe barrel 150 or plunger 160. As a result, needle cover 120 covers needle 140 and the bevel (tip) of needle 140 may be aligned with needle plug 130, into which a plug material, such as clay, may be placed. Relative axial movement between needle 140 and needle plug 130 may move a distal end of the needle into the plug material.

In some embodiments, luer adapter 110 and a hub of the needle 140 may be keyed to assume a particular orientation upon coupling. For example, a needle hub of needle 140 may have a protrusion, such as needle hub portion 111; and the luer adapter 110 may have a slot that receives the protrusion. As a result, when needle 140 and luer adapter 110 are coupled, needle 140 and luer adapter 110 may assume a single orientation. The single orientation may place needle cover 120 extending at an angle between 45 degrees and 90 degrees, e.g., 90 degrees, from a central axis of the needle. The single orientation may be used by the caregiver to assure that the caregiver is approaching the patient with needle 140 in the correct orientation. The caregiver may insert needle 140 into the patient with the bevel of needle 140 facing upward so that the distalmost point of needle 140 (i.e., the sharpest point of needle 140) is on the bottom side, closest to the patient, to provide better entry into the patient. The orientation of needle cover 120, which is at an angle (between 45 degrees and 90 degrees) from the bevel face of needle 140, provides a quick indication to the caregiver that needle 140 is properly oriented.

In addition, in some examples when needle 140 is being used to draw blood from an artery of a patient, needle cover 120 may extend at a 45-90 degree angle from a central axis of plunger 160, the syringe barrel 150, and the needle 140 (by the operation of the hinge portion 112). As a result, needle cover 120 does not interfere with the insertion of needle 140 into the patient, as needle cover 120 is angled away from the patient's skin.

As described, arterial syringe assembly 100 may be operated single-handedly. In some examples after blood or fluid is drawn, needle cover 120 may be closed over needle 140. Needle cover 120 may be closed by pressing the needle cover against a hard surface, such as a bed rail, a wall, a table, or a chair, until the needle snaps into place with an engagement tab of the cover. Similarly, the evacuation of air from the syringe barrel 150, by use of the hydrophobic filter 170, can be performed using a single hand, as the caregiver applies pressure to plunger 160 to push plunger 160 into syringe barrel 150. The pressure may be applied by stretching a finger of the hand holding arterial syringe assembly 100 to plunger 160 or by pushing the plunger against a hard surface.

As shown in FIG. 1, syringe barrel 150 may be transparent or translucent. Because of the transparent or translucent nature of syringe barrel 150, the caregiver may be able to see, through syringe barrel 150, plunger 160 and/or hydrophobic filter 170. Thus, the caregiver may view the blood or fluid drawn from the patient and stored within the syringe barrel and may be able to visually verify that arterial syringe assembly 100 is correctly arranged and operated. In some examples, arterial syringe assembly 100 comprises housing 180. Housing 180 may include a proximal end coupled to syringe barrel 150, luer adapter 110 with hinge portion 112, and a distal end coupled to needle 140 via needle hub portion 111.

FIG. 2A illustrates a side view of an example arterial syringe assembly 200, according to some aspects of the disclosure. FIG. 2B illustrates a bottom view of an example arterial syringe assembly 250, according to some aspects of the disclosure. As shown, arterial syringe assemblies 200 and 250 include luer adapter 210, needle cover 220, and needle plug 230 that are consistent with luer adapter 110, needle cover 120, and needle plug 130 of FIG. 1. Needle plug 230 may be coupled to needle cover 220. Luer adapter 210 has first cover hinge portion 212 that couples with second cover hinge portion 222 at one end of needle cover 220 that is opposite to another end of needle cover 220 that is coupled to needle plug 230. In some examples, first cover hinge portion 212 and second cover hinge portion 222 together make up hinge portion 112 shown in FIG. 1. In some examples, second cover hinge portion 222 snaps in first cover hinge portion 212 to make up the hinge portion. In some embodiments, luer adapter 210 connects between a distal end of syringe barrel 150 and a needle or needle hub (not shown in FIGS. 2A-2B). Needle cover 220 also has locking tab 224 which, when connected to needle plug 230, locks needle plug 230 into place.

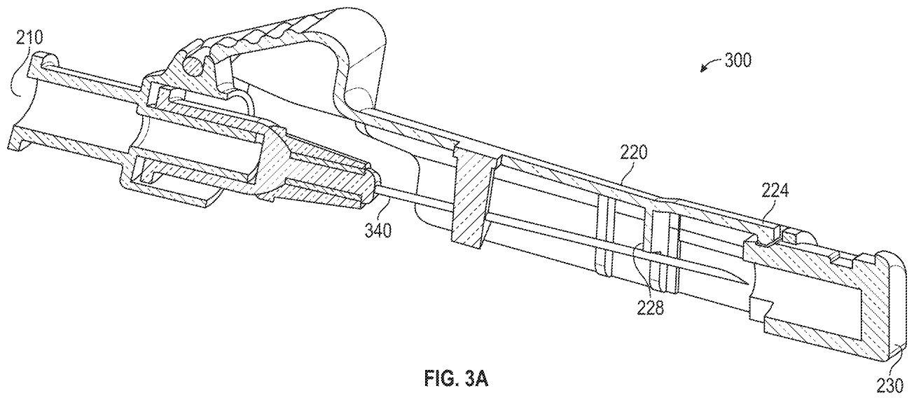

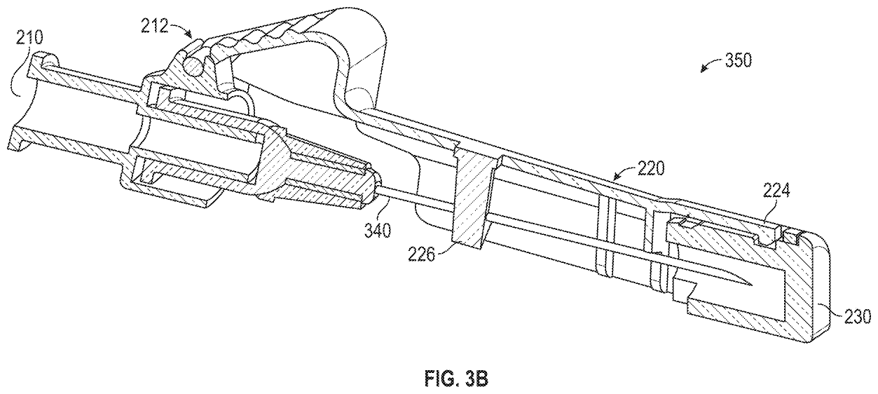

FIG. 3A illustrates an example arterial syringe assembly with a needle locked and ready for shut off, according to some aspects of the disclosure. FIG. 3A shows arterial syringe assembly 300 that is consistent with arterial syringe assembly 200 of FIG. 2A. Arterial syringe assembly 300 includes luer adapter 210, needle cover 220, and needle plug 230 that are connected with needle 340 inside needle cover 220. Locking tab 224 for needle plug 230 is in a first (outer) stage position, as needle plug 230 has not yet been pushed proximally into needle cover 220. Needle stop 228 of needle cover 220 ensures that the needle 340 is positioned at a proper angle relative to needle cover 220. In some examples, a caregiver (e.g., a medical professional) may push needle plug 230 into the needle cover 220 to fully lock the arterial syringe assembly—to place the arterial syringe assembly 100 into the configuration of FIG. 3B, discussed below.

FIG. 3B illustrates the fully locked and shut off configuration of an example arterial syringe assembly, according to some aspects of the disclosure. FIG. 3B shows arterial syringe assembly 350 that is consistent with arterial syringe assembly 300 of FIG. 3A. As shown, arterial syringe assembly 350 includes luer adapter 210, the needle cover 220, and the needle plug 230 are connected with the needle 340 inside the needle cover 220. In some examples as shown in FIG. 3B, locking tab 224 for the needle plug 230 is in a final (inner) stage position and needle plug 230 is pushed into needle cover 220 and over needle 340. Needle plug 230 has been locked into needle cover 220. In some embodiments, needle-locking tab 226 of needle cover 220 locks the needle 340 into place.

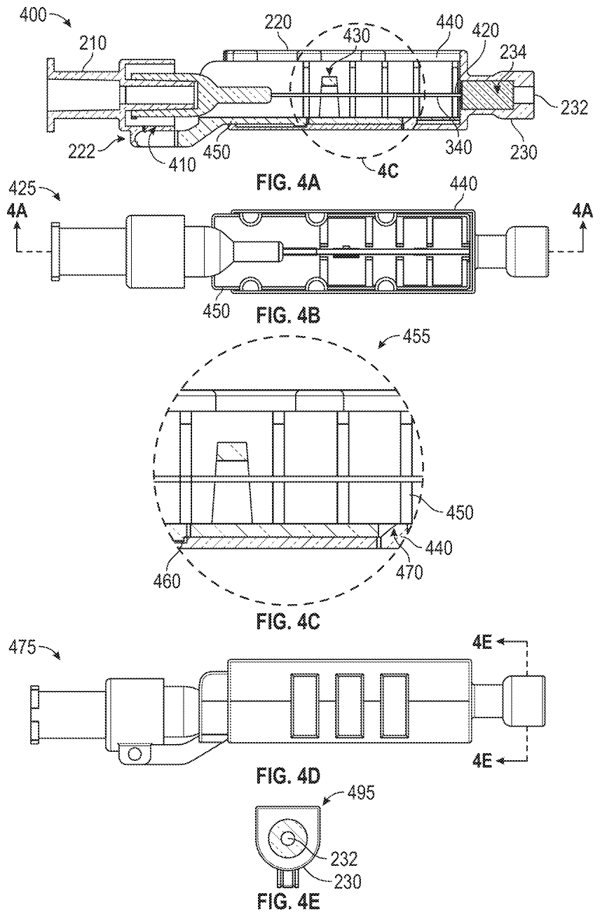

FIG. 4A illustrates an interior view of an example arterial syringe assembly, according to some aspects of the disclosure. FIG. 4A shows diagram 400 of an arterial syringe assembly that is consistent with arterial syringe assembly 200 of FIG. 2A. The arterial syringe assembly includes luer adapter 210, needle cover 220, and needle plug 230. In some embodiments, needle plug 230 is included in needle cover 220. As shown, needle cover 220 includes outer sleeve 440 and inner sleeve 450. Needle plug 230 may include internal chamber 234 and inlet port 232. In some examples, needle 340 accesses internal chamber 234 through hole 420 or an aperture provided for the needle. As shown, a distal end of needle cover 220 includes inlet port 232 to insert, e.g., to inject, a plug material. The distal end of needle cover 220 is described with respect to FIG. 4E.

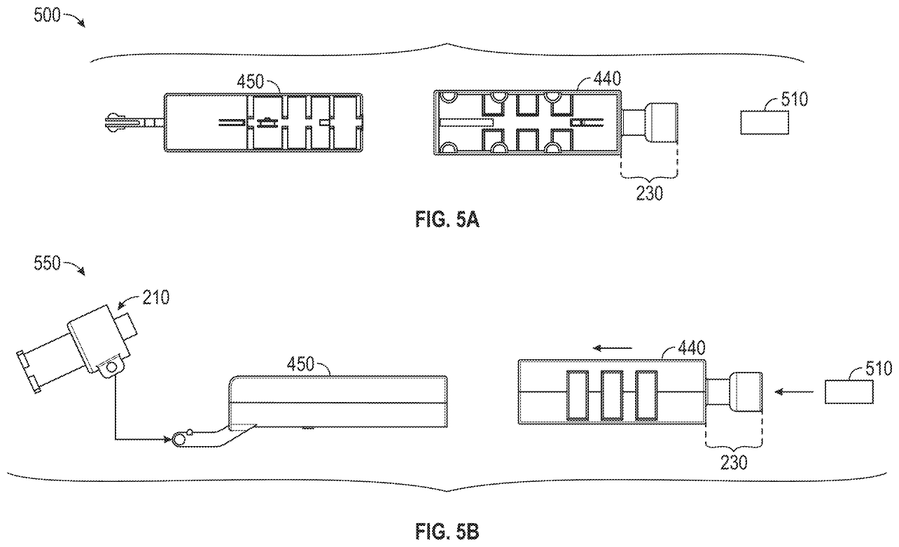

In some examples as shown in FIG. 4A, needle cover 220 includes second cover hinge portion 222. Second cover hinge portion 222 may snap into first cover hinge portion 212 of the luer adapter 210 shown in FIGS. 2A and 3B to put needle cover 220 into place. The needle cover 220 also includes needle-locking tab 430, which secures the needle when the needle is forced into needle cover 220 of arterial syringe assembly. As further shown in FIG. 4A, stop surface 410 of needle cover 220 keeps the needle parallel to the walls of needle cover 220 when the needle plug is snapped into needle-locking tab 430. FIG. 4B illustrates a top view of an example arterial syringe assembly, according to some aspects of the disclosure. Diagram 425 of FIG. 4B includes luer adapter 210, needle cover 220, and needle plug 230. In some examples, FIG. 4A is a cross-sectional view along surface A-A of FIG. 4B. FIG. 4C illustrates a detailed view of locking tabs of the example arterial syringe assembly of FIG. 4A, according to some aspects of the disclosure. As shown in FIG. 4C, the needle cover includes outer sleeve 440 and inner sleeve 450. Outer sleeve 440 and inner sleeve 450 connect to one another through locking tabs, i.e., a locking tab 460 on the inner sleeve 450 and a locking tab 470 on the outer sleeve 440. Outer sleeve 440 and inner sleeve 450 are described with respect to FIGS. 5A and 5B.

FIG. 4D illustrates a side view of an example arterial syringe assembly having an inner sleeve and an outer sleeve, according to some aspects of the disclosure. FIG. 4E illustrates a cross sectional view of a distal end of an example arterial syringe assembly, according to some aspects of the disclosure. In some examples, diagram 495 of FIG. 4E is a cross-sectional view along surface E-E of FIG. 4D. Diagram 495 shows inlet port 232 of the distal end of needle cover 220 that may be used of inserting the plug material into internal chamber 234. The plug material may be clay or some other plug material. Internal chamber 234 may retains plug material for needle shut off configuration.

FIG. 5A illustrates a top view of components of an example arterial syringe assembly, according to some aspects of the disclosure. Diagram 500 of FIG. 5A illustrates inner sleeve 450, outer sleeve 440, needle plug 230, and plug material 510. FIG. 5B illustrates a top view of components of an example arterial syringe assembly, according to some aspects of the disclosure. Diagram 550 of FIG. 5B illustrates luer adapter 210, inner sleeve 450, outer sleeve 440, needle plug 230, and plug material 510. Plug material 510 may be placed inside needle plug 230 to plug the needle 340 shown in FIGS. 3A, 3B, and 4A. Plug material 510 may be clay or some other plug material that may occlude the distal tip of the needle. Diagrams 500 and 550 of FIGS. 5A and 5B are consistent with diagrams 400, 425, and 475 of FIGS. 4A, 4B, and 4C.

As illustrated in FIGS. 4A-4D and 5A-5B, needle cover 220 may comprise of an inner sleeve 450 and an outer sleeve 440. Inner sleeve 450 and outer sleeve 440 are separate components that couple together, as shown in FIGS. 4A-4D. The inner sleeve 450 and the outer sleeve 440 may connect with one another through at least one locking tab of the outer sleeve, e.g., locking tab 470 and at least one locking tab of the inner sleeve, e.g., locking tab 460. In some embodiments, needle plug 230 may reside at a distal end of outer sleeve 440. In some embodiments, inner sleeve 450 is rotationally coupled through the hinge to luer adapter 210 and permits outer sleeve 440 to slidingly couple with inner sleeve 450. In some embodiments, needle cover 220 may be a single unit that does not include multiple sleeves.

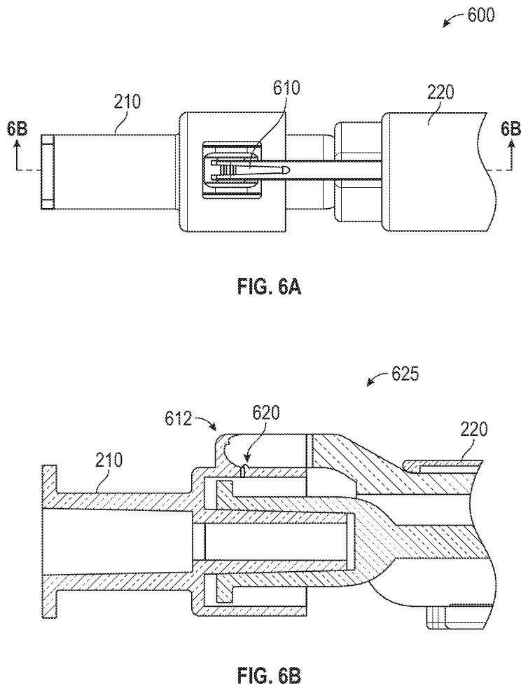

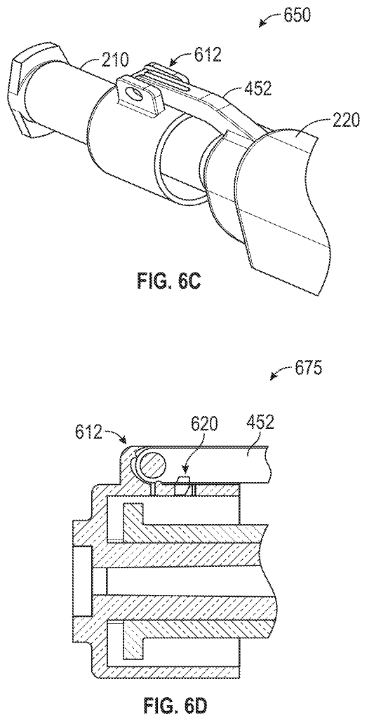

FIGS. 6A-6D illustrate components of a hinge of an example arterial syringe assembly, according to some aspects of the disclosure. FIGS. 6A-6D illustrate operations of hinge portion 612, which locks inner sleeve 450 of the needle cover 220 to luer adapter 210. Hinge portion 612 may correspond to first cover hinge portion 212 and/or second cover hinge portion 222 in FIGS. 2A-2B. FIG. 6A illustrates a top view 600 and FIG. 6B illustrates a cross section view 625 of luer adapter 210, hinge portion 612, and needle cover 220. Cross section view 625 also shows locking tab 620. As shown in FIG. 6A, hinge portion 612 is designed to snap into locking tab 620 of luer adapter 210. FIG. 6C illustrates an angled view 650 of luer adapter 210, hinge portion 612, and needle cover 220. As shown in FIG. 6C, the hinge portion 612 holds together luer adapter 210 and the needle cover 220. FIG. 6D illustrates a close-up view 675 of luer adapter 210 having hinge portion 612 and locking tab 620. In some embodiments, locking tab 620 may be a dual positive locking tab, which resists tampering when fully engaged.

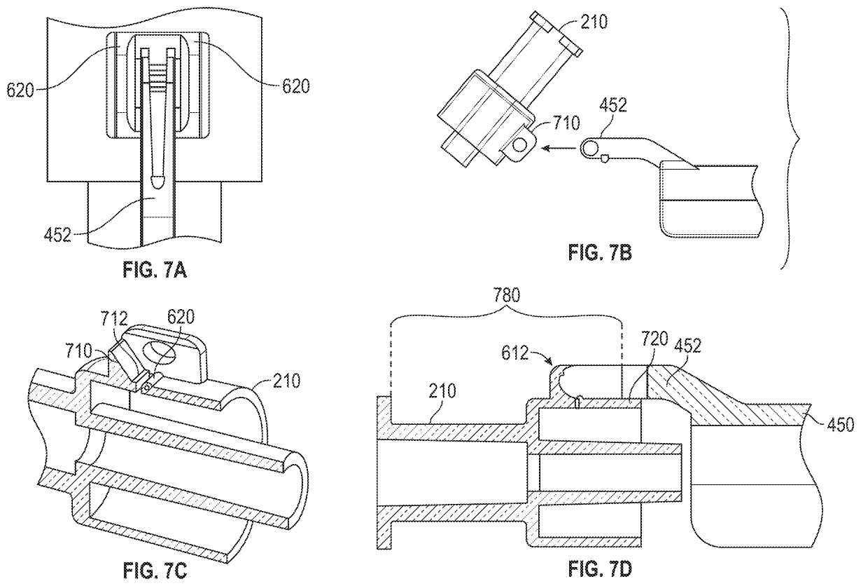

FIGS. 7A-7D illustrate components of an example arterial syringe assembly, according to some aspects of the disclosure. FIG. 7A illustrates locking tab 620 of luer adapter 210 and inner sleeve arm 452 of inner sleeve 450. As shown in FIG. 7A, inner sleeve arm 452 creates a slight side force on locking tabs 620, holding hinge portion 612 and causing the luer adapter 210 to be held in place. FIG. 7B illustrates luer adapter 210 and inner sleeve arm 452. As shown, luer adapter 210 has angle stop 710, into which the inner sleeve arm 452 may be frictionally fit to create a hinge consistent with hinge portion 612 of FIGS. 6A-6D.

FIG. 7C illustrates a detailed view of luer adapter 210. As shown, angle stop 710 of luer adapter 210 controls the amount of angle travel allowed by inner sleeve arm 452 (not shown in FIG. 7C). Adjacent to angle stop 710 is cut out 712. Cut out 712 creates a spring clip that applies a positive force to inner sleeve arm 452 after the inner sleeve arm 452 is installed into angle stop 710.

FIG. 7D illustrates a detailed view of housing 780 that is consistent with housing 180 of FIG. 1. Housing 780 includes luer adapter 210 coupled to hinge portion 612. Inner sleeve 450 is coupled through inner sleeve arm 452 to luer adapter 210. As shown in FIG. 7D, stop surface 720 that is consistent with stop surface 410 of FIG. 4A, is used when the needle is in its locked position. Stop surface 720 may be used for the rotational travel of the inner sleeve 450 about the hinge portion 612. The hinge portion 612 engages the top of the luer adapter 210 when the hinge portion 612 is in the locked position. The engagement of inner sleeve 450 with luer adapter 210 provides a stop to limit rotational travel of the inner sleeve 450 about hinge portion 612.

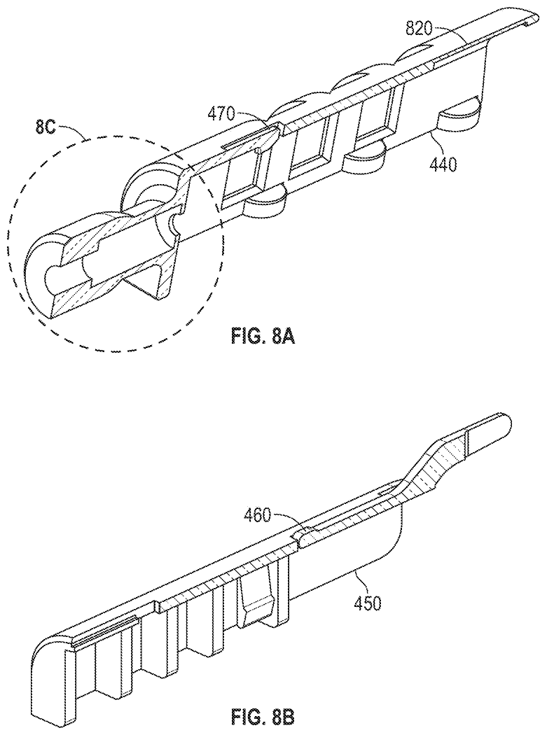

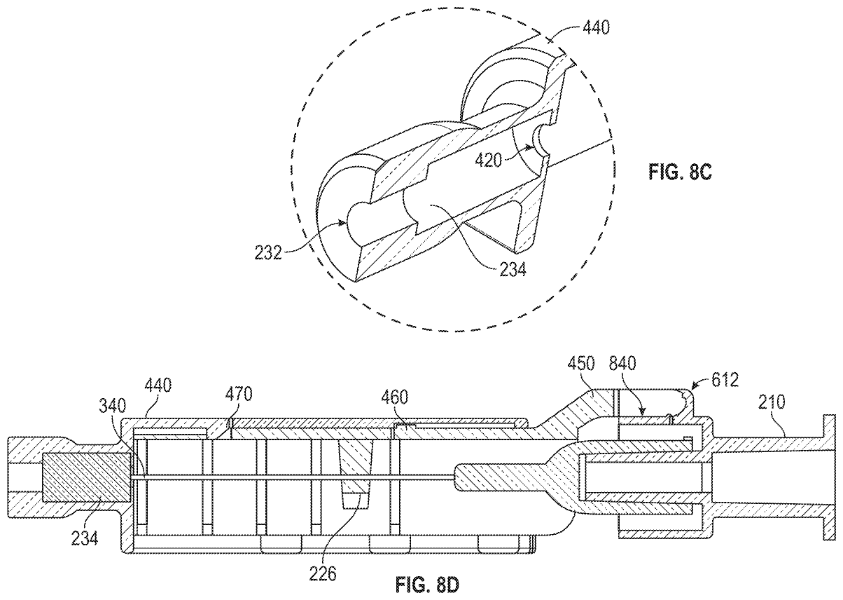

FIG. 8A-8D illustrate components of an example arterial syringe assembly, according to some aspects of the disclosure. FIG. 8A illustrates an inside view of outer sleeve 440 of FIG. 5B, which includes a locking tab 470 for connecting to inner sleeve 450 and also includes slot 820 for installing the inner sleeve and allowing inner sleeve 450 to move into the outer sleeve 440. FIG. 8B illustrates an inside view of inner sleeve 450 of FIG. 5B, which also includes locking tab 460 for connecting to outer sleeve 440.

FIG. 8C illustrates the distal end of the outer sleeve 440 of FIG. 8A in detail. FIG. 8C corresponds to a section of FIG. 8A marked by a circle C. FIG. 8C shows a distal end of needle cover 220 that includes an inlet port 232 to inject the plug material. In some examples, inlet port 232 leads to the internal chamber 234 that retains the plug material that is injected through inlet port 232. Hole 420 of internal chamber 234 is provided for the needle (not shown in FIG. 8C) to access the plug material inside the internal chamber 234.

FIG. 8D illustrates a fully locked and shut off state of the arterial syringe assembly. As shown in FIG. 8D, locking tab 470 of the outer sleeve 440 locks the outer sleeve 440 to the inner sleeve 450, after the outer sleeve 440 and the inner sleeve 450 have been rotated around the hinge to align with the needle, and the outer sleeve 440 has been withdrawn or pressed proximally to slide over the inner sleeve 450. Locking tab 460 of the inner sleeve 450 locks the inner sleeve 450 to the outer sleeve 440. The needle 340 is locked into place by the needle-locking tab 226 of the inner sleeve 450. In some embodiments, luer adapter 210 includes a stop surface 840 to control the angle of locking of luer adapter 210 to the inner sleeve 450 or the outer sleeve 440. Stop surface 840 connects the inner sleeve 450 to the hinge portion 612 and engages the outer surface of the luer adapter 210 to prevent further rotation of the inner sleeve 450. Stop surface 840 ensures that the inner sleeve 450, when locked, is in the proper orientation for sliding the outer sleeve 440 axially in the proximal direction in order to seal the distal end of needle 340. As shown, needle 340 extends into the internal chamber 234, which is filled with plug material, which plugs needle 340.

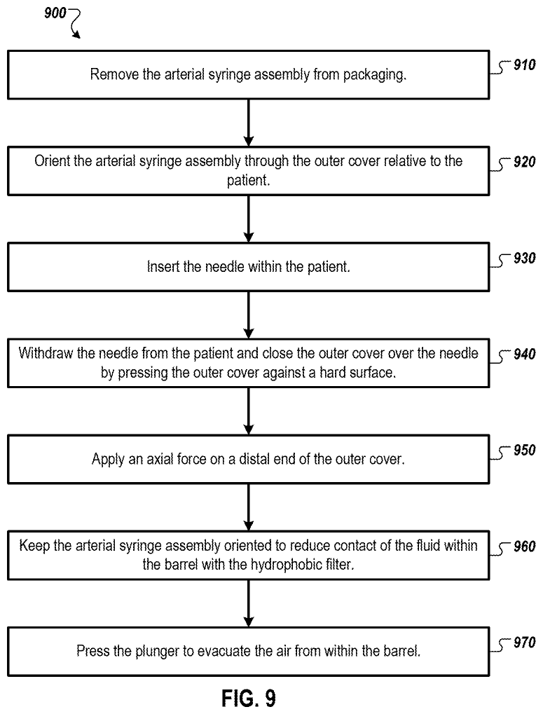

FIG. 9 illustrates a flow diagram of an example process of operating an arterial syringe assembly, according to some aspects of the disclosure. Notably, one or more steps of process 900 described herein may be omitted, performed in a different sequence, and/or combined with other processes for various types of applications contemplated herein. Process 900 can be performed to operate arterial syringe assembly 100 of FIG. 1. Prior to beginning the process 900, the arterial syringe assembly may be delivered to a caregiver from the manufacturer (possibly via an intermediary, such as an online store or a brick-and-mortar store) in tamper-proof packaging.

The process 900 begins at step 910, where the caregiver removes the arterial syringe assembly from the packaging. The packaging may be plastic packaging and may include additional protective material (e.g., additional plastic or cotton covering) around the needle. The packaging may include a warning label to prevent the caregiver from opening or tampering with the arterial syringe assembly before use.

In step 920, the caregiver orients the arterial syringe assembly through the needle cover relative to the patient. The needle may be inserted directly into the patient at an angle of approximately 30 degrees relative to the patient's skin at the point of injection or at another angle. The needle cover may be oriented to make an angle of at least 45 degrees, e.g., 90 degrees, relative to the needle bevel surface, so as not to disturb the patient's skin and the needle as the needle is injected through the patient's skin into the patient's artery. The needle cover may point away from the patient's skin. Prior to inserting the needle into the patient, the caregiver may pull back the plunger to loosen the plunger, as the plunger may have become tightly fitted within the syringe barrel since the manufacture of the arterial syringe assembly.

In step 930, the caregiver inserts the needle within the patient. The needle is then used to draw blood or fluid from the patient's artery into the syringe barrel of the arterial syringe assembly. During the drawing of blood or fluid, the caregiver may slowly pull back the plunger to reduce pressure within the syringe barrel and, thereby, cause blood or fluid to flow into the syringe barrel. In some instances, the arterial blood pressure is sufficient to evacuate air from the syringe barrel through the hydrophobic filter such that withdrawal of the needle while blood is being drawn into the syringe barrel is not necessary. The syringe barrel may be a graduated cylinder with markings on the side to allow the caregiver to visually determine an amount of blood or fluid that has been drawn and to remove the needle after a predefined amount of blood or fluid has been drawn. In instances when the plunger is drawn proximally, the plunger may be withdrawn with the same hand that is holding the arterial syringe assembly by stretching a thumb or forefinger of that hand to the plunger and pushing that thumb or forefinger away from the needle.

In step 940, the caregiver withdraws the needle from the patient after noticing that a predefined amount of blood or fluid has been drawn from the patient's artery. The caregiver closes the needle cover over the needle, causing the tip of the needle to extend into the plug material (e.g., clay) within the needle plug. The closing of the needle cover over the needle may be accomplished with the single hand that is holding the needle by pressing the needle cover against a hard surface until the needle cover snaps into place over the needle. The hard surface may be any hard surface available near the caregiver, such as a bed rail, a wall, a table or a chair. In some examples, the caregiver may use his/her other hand for a different task, such as applying a bandage to the patient.

In step 950, the caregiver applies an axial force on a distal end of the needle cover. The axial force may be applied using a single hand, for example, by pushing the distal end of the needle cover against a hard surface. According to some embodiments, the axial force causes the distal end to slide inside the cover to occlude the tip of the needle with the needle plug. According to other embodiments, the axial force causes the distal end of an outer sleeve (e.g., outer sleeve 440) of the needle cover to slide over an inner sleeve (e.g., inner sleeve 450) of the needle cover to occlude the tip of the needle with the needle plug.

In step 960, the caregiver keeps the arterial syringe assembly oriented to reduce contact of the fluid within the syringe barrel with the hydrophobic filter. For example, the caregiver may hold the arterial syringe assembly such that the hydrophobic filter is at the top of the syringe barrel (relative to the Earth or gravitational forces) and the fluid is pulled to the bottom of the syringe barrel by gravity. This orientation of the arterial syringe assembly may be accomplished by rotating the arterial syringe assembly using a single hand.

In step 970, the caregiver presses the plunger into the syringe barrel to evacuate the air from within the syringe barrel. When pressing the plunger, the hydrophobic filter at the distal end of the plunger allows air to escape through the hydrophobic filter, while preventing the escape of fluid. Neither air nor fluid escapes through the needle because the tip of the needle is occluded with the needle plug, which includes a plugging material, such as clay. After step 970, the process 900 ends.

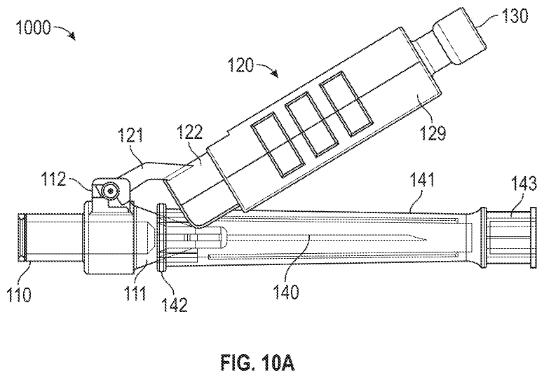

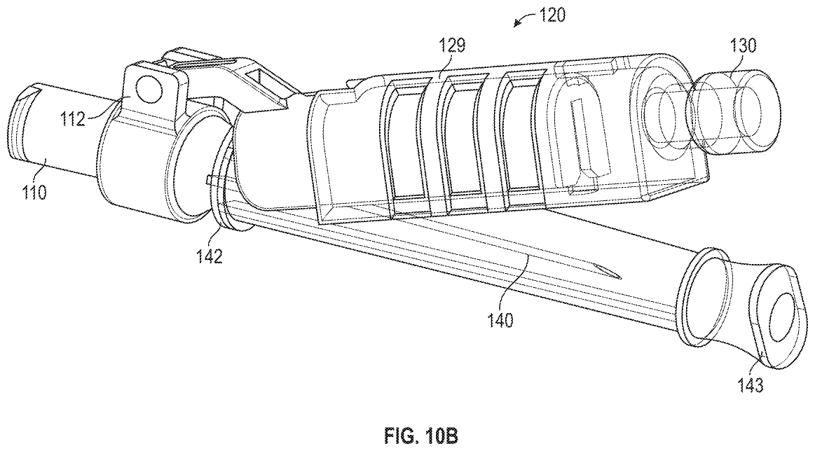

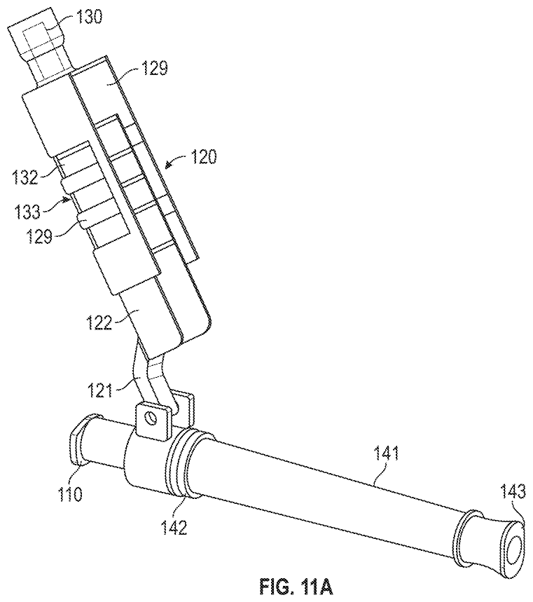

FIG. 10A illustrates a side view of an example arterial syringe assembly, according to some aspects of the disclosure. FIG. 10A shows syringe assembly 1000 that is consistent with a portion of arterial syringe assembly 100 of FIG. 1. Arterial syringe assembly 1000 includes luer adapter 110, needle cover 120, needle plug 130, and needle 140 that are consistent with corresponding elements of arterial assembly 100. Arterial syringe assembly 1000 further includes needle cap 141 that may prevent accidental needle stick. Arterial syringe assembly 1000 may be used similar to Arterial syringe assembly 100 and as described with respect to process 900.

In addition, differences between the arterial syringe assembly 1000 and the arterial syringe assembly 100 are described. In some embodiments, needle cap 141 includes needle cap end 142 and syringe plug end 143 opposite the needle cap end 142. Needle cap 141 may provide a protective covering for needle 140 before use, and includes a hollow portion extending from needle cap end 142 to syringe plug end 143. The needle cap end 142 may interface with a needle hub portion 111 of luer adapter 110. For example, needle hub portion 111 may have one or more ridges and needle cap end 142 may have corresponding grooves that can fit into the ridges. In some embodiments, syringe plug end 143 is shaped like luer adapter 110. Thus, syringe plug end 143 may interface with syringe barrel 150 for sealing the syringe barrel 150 when the luer adapter 110 is removed.

In some embodiments, luer adapter 110 may be formed as a single piece with needle 140. Luer adapter 110 may be integrated with the needle hub portion 111 and the hinge portion 112. Hinge portion 112 may include one or more locking tabs with holes for receiving needle cover 120. Locking tabs are described at least with respect to FIGS. 10B, 11B, and 12B.

FIG. 10B illustrates a side view of an example arterial syringe assembly, according to some aspects of the disclosure. In some examples, FIG. 10B is a different view of FIG. 10A and includes luer adapter 110, needle cover 120, needle plug 130, needle 140, hinge portion 112, needle cap end 142, and syringe plug end 143.

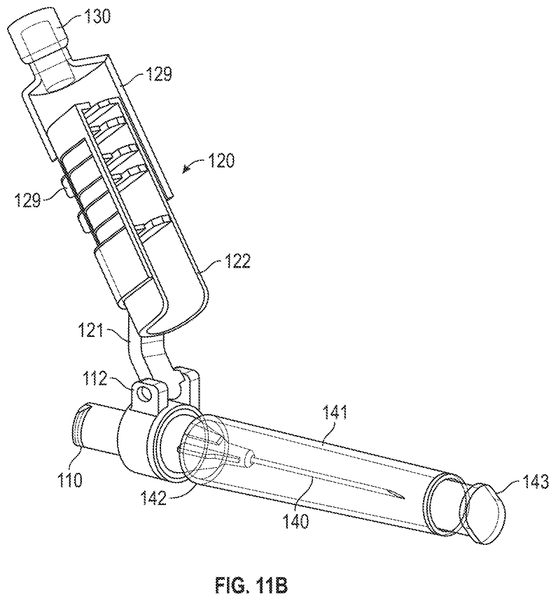

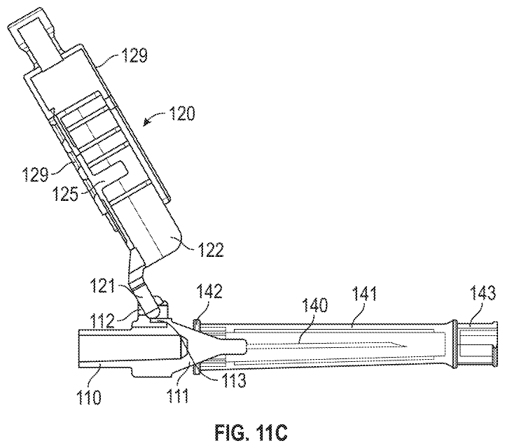

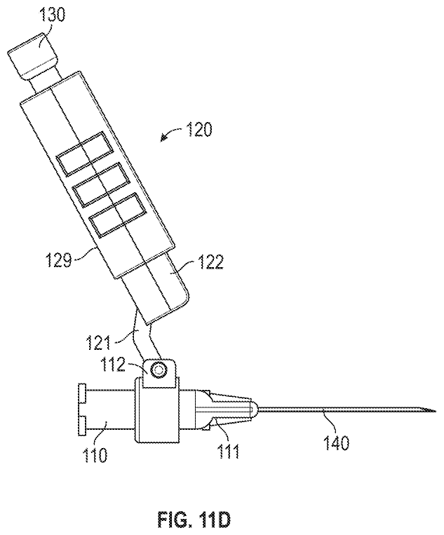

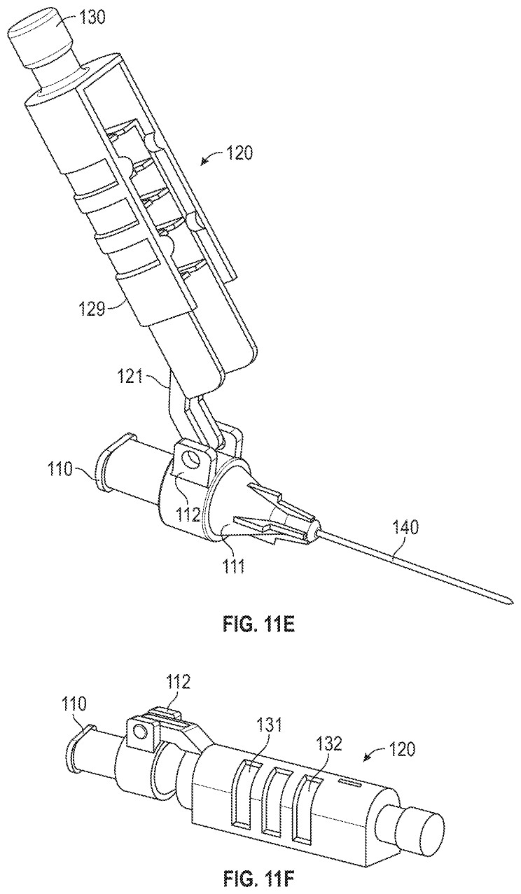

FIGS. 11A-11B illustrate side views of arterial syringe assembly of FIG. 10A with the needle cover in a fully open position, according to some aspects of the disclosure. FIGS. 11A-11B includes needle cover 120, luer adapter 110, needle cap 141 having needle cap end 142 and syringe plug end 143, and needle plug 130. Needle cover 120 has inner sleeve 122 that is consistent with inner sleeve 450 of FIGS. 5A-5B and outer sleeve 129 that is consistent with outer sleeve 440 of FIGS. 5A-5B. Inner sleeve 122 includes one or more arms 121 that are consistent with inner sleeve arm 452. Inner sleeve 122 may interface with the holes of the tabs of the hinge portion 112 to form a hinge about which the needle cover 120 rotates.

In some embodiments, outer sleeve 129 includes one or more windows 133. Windows 133 may function as ridges, which may enhance grip for holding and sliding outer sleeve 129. Windows 133 also provide openings for viewing locked indicator 131 and the unlocked indicator 132. When outer sleeve 129 is in the unlocked position, which may be distal from inner sleeve 122, only unlocked indicator 132 may be visible through windows 133. When outer sleeve 129 is in the locked position, which may be proximate to inner sleeve 122, only locked indicator 131 may be visible through windows 133. Unlocked indicator 132 may be a visual indicator, such as green or other color, pattern, icon, etc., which indicates that outer sleeve 129 is in the unlocked position. The locked indicator 131 may be a visual indicator, such as red or other color, pattern, icon, etc., which indicates that outer sleeve 129 is in the locked position. When in the locked position, the plug material 510 inside the needle plug 130 engages the needle 140.

FIG. 11C illustrates a side cutaway view of the arterial syringe assembly of FIG. 10A, according to some aspects of the disclosure. FIG. 11C includes same components described above for FIGS. 11A-11B. Additionally FIG. 11C shows needle hub portion 111, needle 140, and locking tab 113 of hinge portion 112 that is consistent with lock tab 620 of FIG. 7C.

FIG. 11D-11E illustrate side views of arterial syringe assembly of FIG. 10A, according to some aspects of the disclosure. FIGS. 11D-11E include needle cover 120, luer adapter 110, needle 140 including needle hub portion 111, and needle plug 130. Needle cover 120 has inner sleeve 122 and outer sleeve 129. Inner sleeve 122 includes one or more arms 121 that may interface with the holes of the tabs of the hinge portion 112 to form a hinge about which the needle cover 120 rotates.

FIG. 11F illustrates arterial syringe assembly of FIG. 10A in the locked position, according to some aspects of the disclosure. FIG. 11F additionally shows locked indicator 131 and unlocked indicator 132 that may be visible through windows of needle cover 120. In some embodiments, locked indicator 131 is proximal and unlocked indicator 132 is distal.

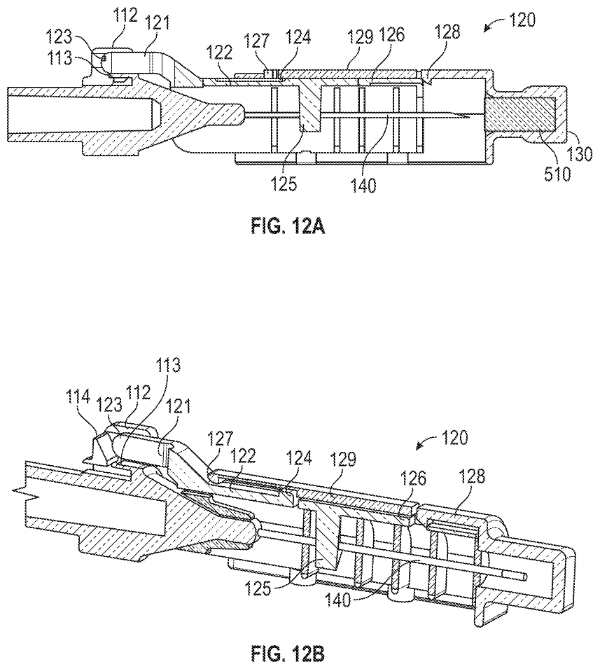

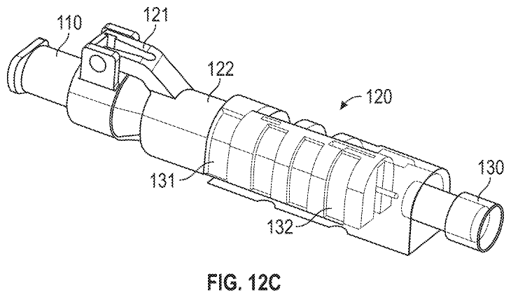

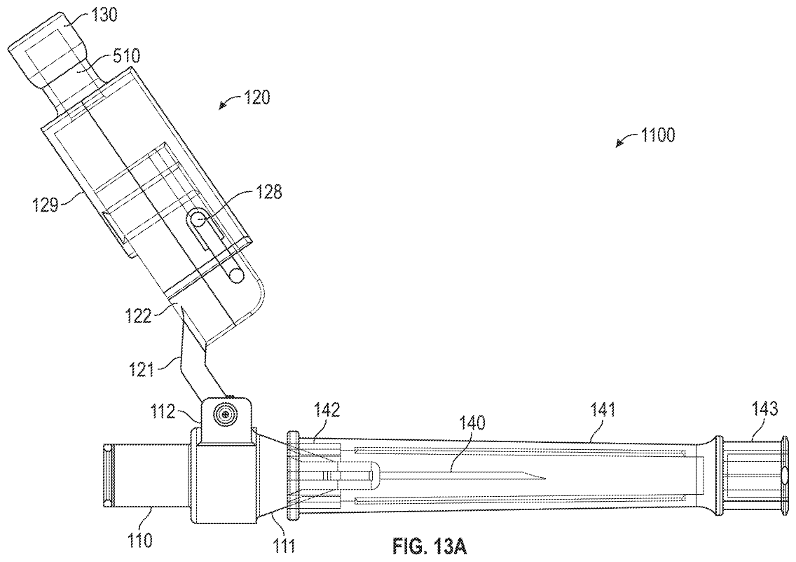

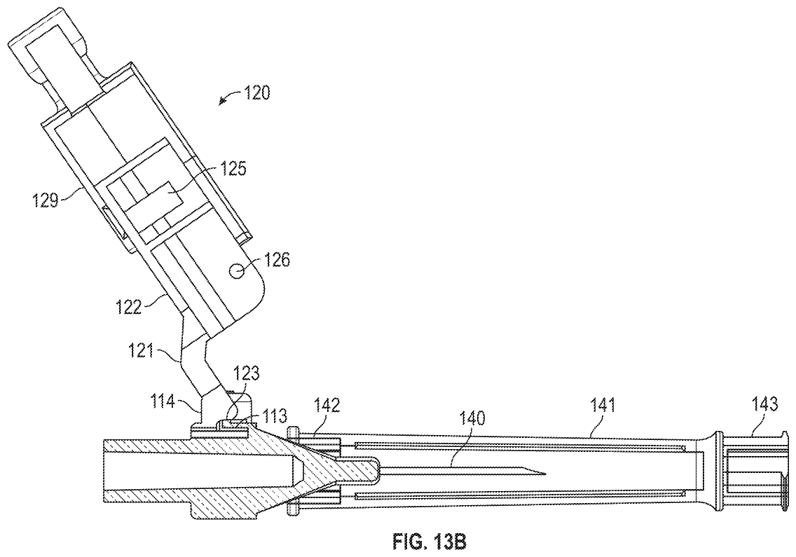

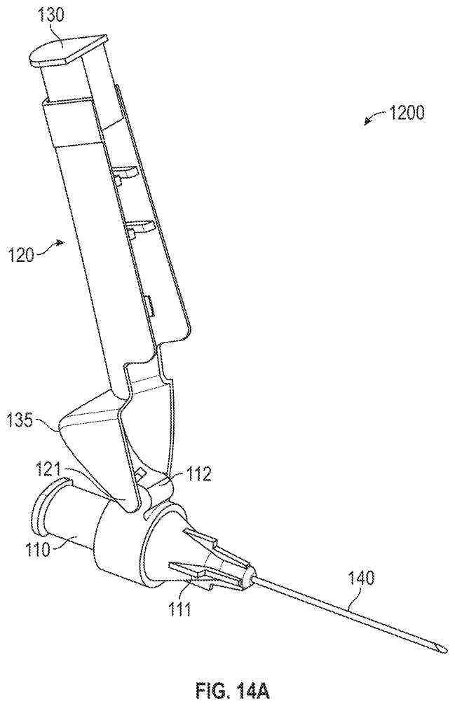

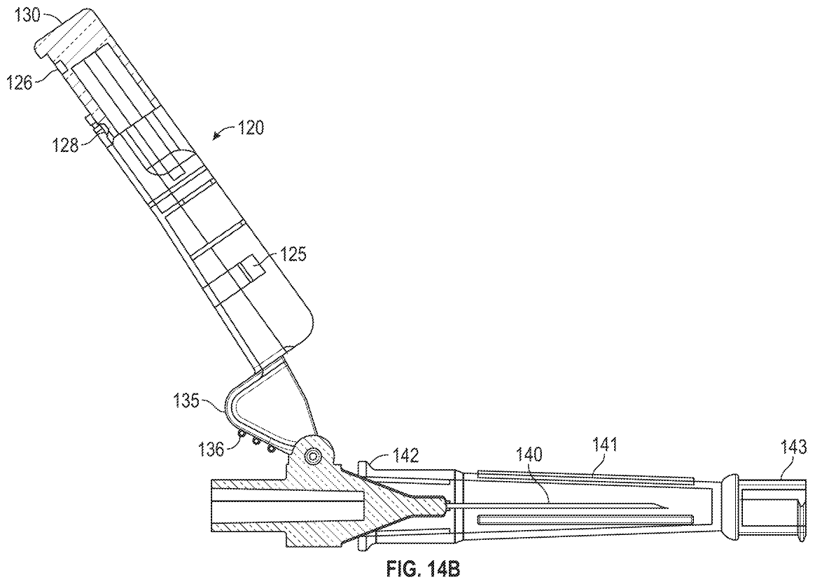

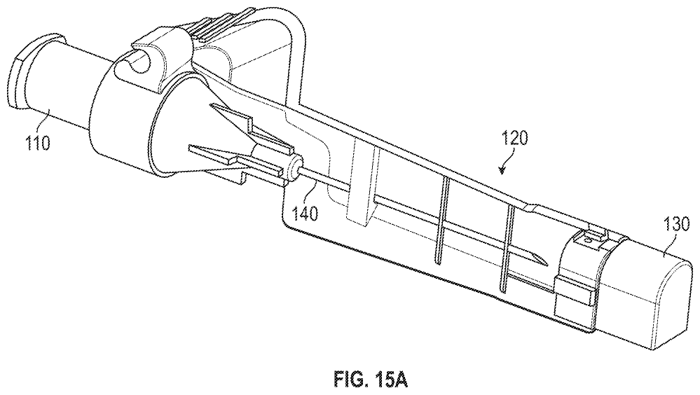

FIG. 12A-12B illustrate side views of arterial syringe assembly of FIG. 10A having the needle cover in a locked position, according to some aspects of the disclosure. As shown in FIG. 12A, hinge portion 112 includes locking tab 113. As shown in FIG. 12B, hinge portion 112 includes locking tab 113 and backstop portion 114. In some embodiments, arms 121 include one or more notches 123, which engage with locking tab 113 of the hinge portion 112. In some examples, notches 123 may be positioned such that notches 123 engage the locking tab 113 at specified angles of the needle cover 120 with respect to the needle 140, e.g., at zero degrees when needle cover 120 is closed. In addition, backstop portion 114 of the hinge portion 112 prevents arms 121 from rotating further beyond backstop portion 114 (angle stop), to establish a maximum position stop for needle cover 120. In some examples, back stop portion 114 is consistent with angle stop 710 of FIG. 7C. In some examples, needle-locking tab 125 which is consistent with needle-locking tab 226 of FIG. 3B engages needle 140 when needle cover 120 is closed, to secure needle 140 and prevent needle cover 120 from opening.

In some examples, outer sleeve 129 slides over the inner sleeve 122. Tab 127 of the outer sleeve 129 engages the inner locking tab 124 to prevent outer sleeve 129 from sliding completely off inner sleeve 122. Tab 128 of the outer sleeve 129 provides friction to engage slot 126 of the inner sleeve 122 when outer sleeve 129 is moved into the locked position to prevent outer sleeve 129 from sliding. In some examples, arterial syringe assembly 1000 operates in two stages. In a first stage, needle cover 120 is rotated towards needle 140, capturing and locking needle 140 to prevent accidental needle stick. The bottom of needle cover 120 is open to allow needle cover 120 to rotate over needle 140. Needle-locking tab 125 slides over and engages needle 140. Inner sleeve 122 may include additional spines to further guide and surround needle 140. In some embodiments, notch 123 engages locking tab 113 to prevent needle cover 120 from rotating out.

In a second stage, the outer sleeve 129 may move from the unlocked position to the locked position. Plug material 510 may be pushed into needle 140. Tab 128 engages slot 126 to lock outer sleeve 129. Unlocked indicator 132 is obscured and locked indicator 131 is made visible through one of windows 133.

FIG. 12C illustrates a side view of arterial syringe assembly of FIG. 10A in the locked position, according to some aspects of the disclosure. As shown unlocked indicator 132 is obscured and locked indicator 131 is made visible to indicate the locked position.

FIGS. 13A-13B illustrate side views of an arterial syringe assembly, according to some aspects of the disclosure. FIGS. 13A and 13B illustrate arterial syringe assembly 1100, which may be consistent with arterial syringe assembly 1000. Arterial syringe assembly 1100 may be used in conjunction with shorter needles than those of the arterial syringe assembly 1000. For example, arterial syringe assembly 1000 may be used with 1 inch needles whereas arterial syringe assembly 1100 may be used with ⅝ inch needles. Arterial syringe assembly 1100 includes luer adapter 110, needle cover 120, needle plug 130, needle 140, and needle cap 141. Needle cover 120 of the arterial syringe assembly 1100 may be shorter than needle cover 120 of the arterial syringe assembly 1000. In some examples, operation of the arterial syringe assembly 1100 may be similar to the two-stage operation of arterial syringe assembly 1000, as described above.

In some embodiments, needle cap 141 includes needle cap end 142 that may interface with needle hub portion 111 of the luer adapter 110. In some embodiments, needle cap 141 includes syringe plug end 143 that is opposite of needle cap end 142 may interface with the syringe barrel 150 for sealing the syringe barrel 150 when luer adapter 110 is removed. Similar to arterial syringe assembly 1000, luer adapter 110 of arterial syringe assembly 1100 may be integrated with needle hub portion 111 and hinge portion 112.

In some embodiments, needle cover 120 includes inner sleeve 122 and outer sleeve 129. Inner sleeve 122 includes one or more arms 121, needle-locking tab 125, and slot 126. Outer sleeve 129 includes tab 128 that is coupled to needle plug 130. Arms 121 may interface with the holes of the tabs of hinge portion 112 to form a hinge about which needle cover 120 rotates. Arms 121 include one or more notches 123, which engage locking tab 113 of the hinge portion 112. For example, notches 123 may be positioned such that notches 123 engage locking tab 113 at specified angles, such as 0 degrees (e.g., when the needle cover 120 is closed and covering the needle 140), and/or 45 degrees. In some embodiments, backstop portion 114 of hinge portion 112 prevents arms 121 from rotating further, to establish a maximum position stop for needle cover 120. Needle-locking tab 125 engages needle 140 when needle cover 120 is closed, to secure needle 140 and prevent needle cover 120 from opening.

In some embodiments, outer sleeve 129 slides over inner sleeve 122. Tab 128 of outer sleeve 129 engages slot 126 of inner sleeve 122 when the outer sleeve 129 is moved into the locked position to prevent outer sleeve 129 from sliding. Slot 126 may be a ball detent. In some embodiments, tab 128 and slot 126 may be on both sides of the needle cover 120. Outer sleeve 129 and inner sleeve 122 may include corresponding rails and/or grooves to guide movement. When outer sleeve 129 is in the unlocked position, which may be distal from inner sleeve 122, plug material 510 inside needle plug 130 is separated from the needle 140. When outer sleeve 129 is in the locked position, which may be proximate to inner sleeve 122, plug material 510 inside the needle plug 130 engages the needle 140.

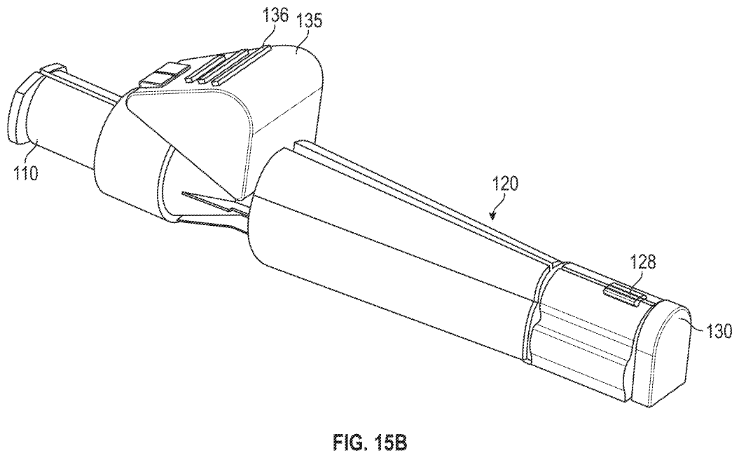

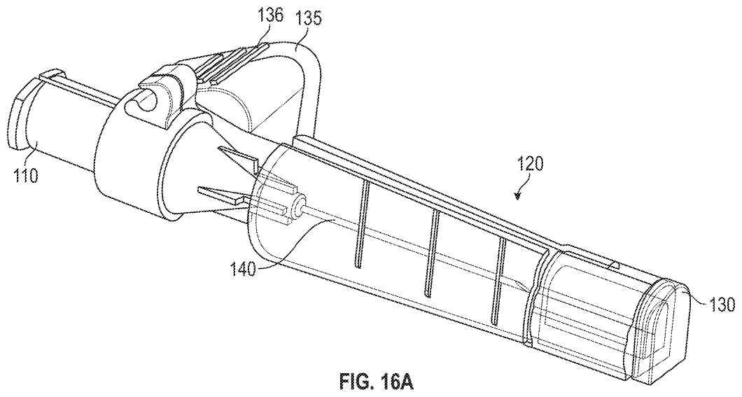

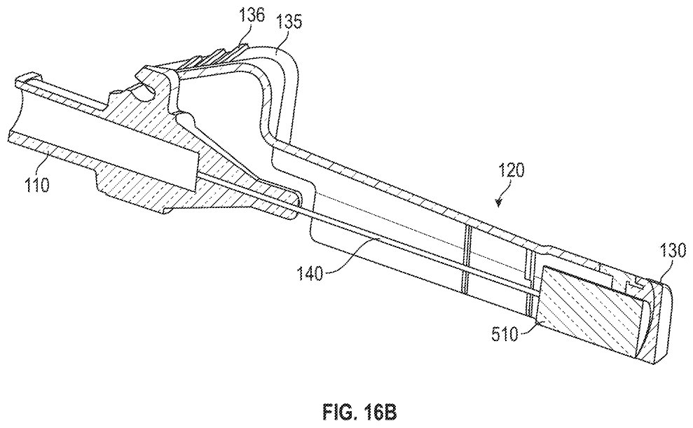

FIGS. 14A-16B illustrate side views of an arterial syringe assembly, according to some aspects of the disclosure. FIGS. 14A-16B illustrate arterial syringe assembly 1200, which may be consistent with arterial syringe assembly 1000. Arterial syringe assembly 1200 includes luer adapter 110, needle cover 120, needle plug 130, needle 140, and needle cap 141. In some examples, operation of the arterial syringe assembly 1200 may be similar to the two-stage operation of the arterial syringe assembly 1000 as described above.

In some embodiments, needle cap 141 includes the needle cap end 142 that may interface with needle hub portion 111 of luer adapter 110. Needle cap 141 may also include syringe plug end 143 opposite the needle cap end 142 that may interface with syringe barrel 150 for sealing syringe barrel 150 when luer adapter 110 is removed. Similar to arterial syringe assembly 1000, luer adapter 110 of arterial syringe assembly 1200 may be integrated with needle hub portion 111 and hinge portion 112.

In some embodiments, needle cover 120 includes the inner sleeve 122 that is coupled to needle plug 130. In some embodiments, one of inner sleeve 122 or outer sleeve 129 includes needle plug 130. Inner sleeve 122 may include one or more arms 121, needle-locking tab 125, and tab 128. Needle plug 130 includes slot 126 and plug material 510. Arms 121 may interface with the holes of the tabs of hinge portion 112 to form a hinge about which needle cover 120 rotates. Needle cover 120 may also include a shoulder 135 having one or more ridges 136. Shoulder 135 provides a surface that may be pushed by hand or a finger. Ridges 136 provide a gripped surface for shoulder 135. In addition, shoulder 135 acts as a backstop, to establish a maximum position stop for needle cover 120. Needle-locking tab 125 engages needle 140 when the needle cover 120 is closed, to secure needle 140 and prevent needle cover 120 from opening.

In some embodiments, needle plug 130 slides within inner sleeve 122. When in the locked position, tab 128 engages the slot 126 of needle plug 130. In the locked position, plug material 510 is pushed into needle 140.

In some embodiments, arterial syringe assembly 1200 operates in two stages. In the first stage, needle cover 120 is rotated towards needle 140, capturing and locking needle 140 to prevent accidental needle stick. The bottom of needle cover 120 is open to allow needle cover 120 to rotate over needle 140. Needle-locking tab 125 slides over and engages needle 140. Inner sleeve 122 may include additional spines to further guide and surround needle 140.

In the second stage, needle plug 130 is moved from the unlocked position to the locked position. Plug material 510 is pushed into needle 140. The tab 128 engages the slot 126 to lock needle plug 130.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “module for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

What is claimed is:

1. An arterial syringe assembly comprising:
a housing comprising (i) a proximal end, (ii) a distal end, and (iii) a hinge, the proximal end of the housing is configured to couple with a distal end of a syringe barrel, and the distal end of the housing is configured to couple with a needle;
a needle cover coupled with the housing at the hinge and being configured to rotate about the hinge to position the needle cover relative to the needle and to rotate to an orientation that is axially aligned with the needle such that the needle is received within the needle cover;
a needle plug coupled to the needle cover at a distal end of the needle cover and comprising a plug material, the needle plug being configured to slide axially relative to the needle to advance proximally when the needle cover is axially aligned with the needle such that a tip of the needle extends within the plug material; and
a hydrophobic filter positioned within the syringe barrel configured to evacuate air through a proximal end of the syringe barrel after the tip of the needle extends within the plug material.

2. The arterial syringe assembly of claim 1, further comprising a plunger residing within the syringe barrel, wherein the plunger is movable along a central axis of the syringe barrel, and wherein the hydrophobic filter is coupled with a distal end of the plunger.

3. The arterial syringe assembly of claim 1, wherein the hydrophobic filter is configured to evacuate air from the syringe barrel while impeding transmission of fluid past the hydrophobic filter.

4. The arterial syringe assembly of claim 1, wherein said arterial syringe assembly is configured to be operated single-handedly by a caregiver to draw fluid from an artery of a patient and, after drawing the fluid, to seal the needle within the needle cover and the needle plug.

5. The arterial syringe assembly of claim 1, wherein the needle cover is further configured to rotate about the hinge to at least 45 degrees away from the needle, such that the needle may be injected into a patient without interference from the needle cover.

6. The arterial syringe assembly of claim 1, wherein the plug material comprises clay.

7. The arterial syringe assembly of claim 1, wherein the needle plug is configured to slide axially relative to the needle cover to advance proximally within the needle cover when the needle cover is axially aligned with the needle such that a tip of the needle extends within the plug material.

8. An arterial syringe assembly comprising:
a housing comprising (i) a proximal end, (ii) a distal end, and (iii) a hinge, the proximal end of the housing is configured to couple with a distal end of a syringe barrel, and the distal end of the housing is configured to couple with a needle;
a needle cover coupled with the housing at the hinge and being configured to rotate about the hinge to position the needle cover relative to the needle and to rotate to an orientation that is axially aligned with the needle such that the needle is received within the needle cover;
a needle plug coupled to the needle cover at a distal end of the needle cover and on an outer surface of the needle cover, the needle plug comprising a plug material, and the needle plug being configured to slide along the outer surface of the needle cover to advance proximally within the needle cover when the needle cover is axially aligned with the needle such that a tip of the needle extends within the plug material; and
a hydrophobic filter positioned within the syringe barrel configured to evacuate air through a proximal end of the syringe barrel after the tip of the needle extends within the plug material.

9. The arterial syringe assembly of claim 8, further comprising a plunger residing within the syringe barrel, and wherein the hydrophobic filter is coupled with a distal end of the plunger.

10. The arterial syringe assembly of claim 8, wherein the hydrophobic filter is configured to evacuate air from the syringe barrel while impeding transmission of fluid.

11. The arterial syringe assembly of claim 8, wherein said arterial syringe assembly is configure to be operated single-handedly by a caregiver to draw fluid from an artery of a patient and, after drawing the fluid, to seal the needle within the needle cover and the needle plug.

12. The arterial syringe assembly of claim 8, wherein the needle cover is further configured to rotate about the hinge to between 45 degrees and 90 degrees away from the needle.

13. The arterial syringe assembly of claim 8, wherein the plug material comprises clay.

14. The arterial syringe assembly of claim 8, wherein the syringe barrel is transparent or translucent, allowing a caregiver to see, through the syringe barrel, one or more of a plunger, the hydrophobic filter, and fluid stored within the syringe barrel.

15. The arterial syringe assembly of claim 8, wherein the needle cover comprises an outer sleeve and an inner sleeve, and the outer sleeve comprises the needle plug and is configured to slide axially over the inner sleeve.

16. The arterial syringe assembly of claim 15, wherein the outer sleeve and the inner sleeve are coupled through at least one locking tab of the outer sleeve and at least one locking tab of the inner sleeve.

17. The arterial syringe assembly of claim 15, wherein the needle plug resides at a distal end of the outer sleeve.

18. A method comprising:
providing an arterial syringe assembly comprising (i) a proximal end configured to couple with a distal end of a syringe barrel, (ii) a distal end coupled to a needle, (iii) a hinge coupled with a needle cover, and (iv) a needle plug, which holds a plug material, coupled with the needle cover at a distal end of the needle cover;
rotating the needle cover about the hinge to position the needle cover relative to the needle that is axially aligned with the needle such that the needle is received within the needle cover;
sliding the needle plug axially relative to the needle, when the needle cover is axially aligned with the needle, to advance the needle plug proximally relative to the needle, such that a distal end of the needle extends within and is plugged by the plug material; and
evacuating, via a hydrophobic filter positioned within the syringe barrel, air through a proximal end of the syringe barrel after the distal end of the needle extends within the plug material.

19. The method of claim 18, wherein sliding the needle plug axially relative to the needle comprises sliding the needle plug proximally within the needle cover to advance the needle plug proximally within the needle cover, when the needle cover is axially aligned with the needle, such that a tip of the needle extends within the plug material.

20. The method of claim 18, wherein sliding the needle plug axially relative to the needle comprises sliding an outer surface of the needle cover relative to an inner surface, and wherein the outer surface comprises, at a distal end of the outer surface, the needle plug.
